SEQUENCE LISTING (7 sequences)

SEQ ID NO: 1 - protein, 1497 aa, Homo sapiens
MDVTKKNKRDGTEVTERIVTETVTTRLTSLPPKGGTSNGYAKTASLGGGSRLEKQSLTHGSSGYINSTGSTRGHASTSSYRRAHSPASTLPNSPGSTFERKTHVTRHAYEGSSSGNSSPEYPRKEFASSSTRGRSQTRESEIRVRLQSASPSTRWTELDDVKRLLKGSRSASVSPTRNSSNTLPIPKKGTVETKIVTASSQSVSGTYDATILDANLPSHVWSSTLPAGSSMGTYHNNMTTQSSSLLNTNAYSAGSVFGVPNNMASCSPTLHPGLSTSSSVFGMQNNLAPSLTTLSHGTTTTSTAYGVKKNMPQSPAAVNTGVSTSAACTTSVQSDDLLHKDCKFLILEKDNTPAKKEMELLIMTKDSGKVFTASPASIAATSFSEDTLKKEKQAAYNADSGLKAEANGDLKTVSTKGKTTTADIHSYGSSGGGGSGGGGGVGGAGGGPWGPAPAWCPCGSCCSWWKWLLGLLLTWLLLLGLLFGLIALAEEVRKLKARVDELERIRRSILPYGDSMDRIEKDRLQGMAPAAGADLDKIGLHSDSQEELWMFVRKKLMMEQENGNLRGSPGPKGDMGSPGPKGDRGFPGTPGIPGPLGHPGPQGPKGQKGSVGDPGMEGPMGQRGREGPMGPRGEAGPPGSGEKGERGAAGEPGPHGPPGVPGSVGPKGSSGSPGPQGPPGPVGLQGLRGEVGLPGVKGDKGPMGPPGPKGDQGEKGPRGLTGEPGMRGLPGAVGEPGAKGAMGPAGPDGHQGPRGEQGLTGMPGIRGPPGPSGDPGKPGLTGPQGPQGLPGTPGRPGIKGEPGAPGKIVTSEGSSMLTVPGPPGPPGAMGPPGPPGAPGPAGPAGLPGHQEVLNLQGPPGPPGPRGPPGPSIPGPPGPRGPPGEGLPGPPGPPGSFLSNSETFLSGPPGPPGPPGPKGDQGPPGPRGHQGEQGLPGFSTSGSSSFGLNLQGPPGPPGPQGPKGDKGDPGVPGALGIPSGPSEGGSSSTMYVSGPPGPPGPPGPPGSISSSGQEIQQYISEYMQSDSIRSYLSGVQGPPGPPGPPGPVTTITGETFDYSELASHVVSYLRTSGYGVSLFSSSISSEDILAVLQRDDVRQYLRQYLMGPRGPPGPPGASGDGSLLSLDYAELSSRILSYMSSSGISIGLPGPPGPPGLPGTSYEELLSLLRGSEFRGIVGPPGPPGPPGIPGNVWSSISVEDLSSYLHTAGLSFIPGPPGPPGPPGPRGPPGVSGALATYAAENSDSFRSELISYLTSPDVRSFIVGPPGPPGPQGPPGDSRLLSTDASHSRGSSSSSHSSSVRRGSSYSSSMSTGGGGAGSLGAGGAFGEAAGDRGPYGTDIGPGGGYGAAAEGGMYAGNGGLLGADFAGDLDYNELAVRVSESMQRQGLLQGMAYTVQGPPGQPGPQGPPGISKVFSAYSNVTADLMDFFQTYGAIQGPPGQKGEMGTPGPKGDRGPAGPPGHPGPPGPRGHKGEKGDKGDQVYAGRRRRRSIAVKP

SEQ ID NO: 2 - DNA, 5610 bp, Homo sapiens
aacatttttgaaagatcactcagctttaacacaccttggctgggtctggataaaaaaaaagtgagcactgcaaatttctagaagaaaacatcaggagaagaaagagagagggggatttattcaaagttgtttccaattccttcaaaacctcaaaccaggtggctatggtatggatgtaaccaagaaaaacaaacgagatggaactgaagtcactgagagaattgtcactgaaacagtaaccacaagacttacatccttaccaccaaaaggcgggaccagcaatggctatgctaaaacagcctctcttggtggagggagccggctggagaaacaaagcctgactcatggcagcagcggctacataaactcaactggaagcacacgaggccatgcctccacctctagttacaggagggctcactcacctgcctccactctgcccaactccccaggctcaacctttgaaaggaaaactcacgttacccgccatgcgtatgaagggagctccagtggcaactcttctccggagtaccctcggaaggaatttgcatcttcttcaaccagaggacggagtcaaacacgagagagtgaaattcgagttcgactgcagagtgcgtccccatccacccgatggacagaattggatgatgttaagcgtttgctcaaggggagtcgatcggcaagtgtgagccccacccggaattcctccaacacactccccatccccaagaaaggcactgtggagaccaaaattgtgacagcgagctcccagtcggtgtcaggcacctacgatgcaacgatcctggatgccaaccttccctcccatgtgtggtcctccaccctgcccgcggggtcctccatggggacctatcacaacaacatgacaacccagagctcatccctcctcaacaccaatgcctactctgcgggatcagtcttcggagttccaaacaacatggcgtcctgctcacccactttgcaccctggactcagcacatcctcctcagtgtttggcatgcagaacaatctggcccccagcttgaccaccctgtcccatggcaccaccaccacttccacagcatatggggtgaagaaaaacatgccccagagtcctgcggctgtgaacactggcgtttccacctccgccgcctgcaccacaagtgtgcagagcgatgaccttttgcacaaggactgcaagttcctgatcctagagaaagacaacacacctgccaagaaggagatggagctgctcatcatgaccaaggacagcgggaaggtctttacagcctcccctgccagcatcgctgcaacttctttttcagaagacaccctaaaaaaagaaaagcaagctgcctacaatgctgactcaggcctaaaagccgaagctaatggagacctgaagactgtgtccacaaagggcaagaccaccactgcagatatccacagctacggcagcagtggtggtggtggcagtggaggaggtggcggtgttggtggcgctggcggcggcccttggggaccagcgccagcctggtgcccctgcggctcctgctgcagctggtggaagtggctgctgggcctgctgctcacctggctgctactcctggggctgctcttcggcctcattgctctggcggaggaggtgaggaagctgaaggcgcgtgtggatgagctggagaggatcaggaggagcatactgccctatggggacagcatggatagaatagaaaaggaccgcctccagggcatggcacccgcggcgggagcagacctggacaaaattgggctgcacagtgacagccaggaggagctctggatgttcgtgaggaagaagctaatgatggaacaggaaaatggaaatctccgaggaagccctggccctaaaggtgacatgggaagtccaggccctaaaggagatcgagggttccctgggactccaggtatccctgggcccttgggccacccaggtccacaaggaccaaagggtcaaaaaggcagcgtgggagatcctggcatggaaggccccatgggccagagagggcgagaaggccccatgggacctcgtggtgaggcagggcctcctggatctggagagaaaggggaaagaggggctgctggtgaaccaggtcctcatggcccacctggtgtcccaggttctgtgggtcccaaaggttccagcggctctcctggcccacagggccctccaggtcctgtaggtctccaagggctccgaggtgaagtaggacttcctggtgtcaaaggtgacaaaggaccaatgggaccaccaggacccaaaggtgaccagggtgagaaaggacctcgaggcctcacaggcgagcctggcatgagaggtttgcctggtgctgttggtgagcccggggctaaaggagcaatgggtcctgctggcccagacggacaccaaggcccaagaggtgaacaaggtcttactgggatgcctggaatccgtggcccaccaggaccttctggagacccaggaaagccaggtctcacaggaccccagggacctcagggacttcccggtacccctggccgaccaggaataaaaggtgaaccaggagctccaggcaagatcgtgacttcggaggggtcatcgatgctcactgtcccaggccccccaggacctcctggagccatgggacccccaggacctccaggtgccccaggccctgccggcccagctggtctcccaggacatcaagaagttcttaatttacaaggtcccccaggcccacccggcccacgcgggccaccagggccttccattccaggcccaccaggaccccgaggcccaccaggggagggtttgccaggcccaccaggcccaccaggatcgttcctgtccaactcagaaaccttcctctccggccccccaggcccacctggccccccaggtcccaagggagaccaaggtcccccaggccccagaggacaccaaggcgagcaaggcctcccaggtttctcaacctcagggtccagttctttcggactcaaccttcagggaccaccaggcccacctggcccccagggacccaaaggtgacaaaggtgatccaggtgttccaggggctcttggcattcctagtggtccttctgaagggggatcatcaagtaccatgtacgtgtcaggcccgccagggccccctgggccccctgggcctccgggctctatcagcagctctggccaggagattcagcagtacatctctgagtacatgcagagtgacagtattagatcttacctatccggagttcagggtcccccaggcccacctggtcccccaggacctgtcaccaccatcacaggcgagactttcgactactcagagctggcaagccacgttgtgagctacttacggacttcggggtacggtgtcagcttgttctcgtcctccatctcttctgaagacattctggctgtgctgcagcgggatgacgtgcgtcagtacctacgtcagtacttgatgggccctcggggtccgccagggccaccaggagccagtggagatgggtccctcctgtctttggactatgcagagctgagtagtcgcattctcagctacatgtcgagttctgggatcagcattgggcttcctggtcccccggggccccctggcttgccgggaacctcctatgaggagctcctctccttgctgcgagggtctgaattcagaggcatcgttggacccccaggtcccccgggtccaccagggatcccaggcaatgtgtggtccagcatcagcgtggaggacctctcgtcttacttacatactgccggcttgtcattcatcccaggccctccaggacctcctggtcccccagggcctcgagggcccccgggtgtctcaggagccctggcaacctatgcagctgaaaacagcgacagcttccggagcgagctgatcagctacctcacaagtcctgatgtgcgcagcttcattgttggccccccaggccctcctgggccgcagggaccccctggggacagccgcctcctgtccacggatgcctcccacagtcggggtagcagctcctcctcacacagctcatctgtcaggcggggcagctcctacagctcttccatgagcacaggaggaggtggtgcaggctccctgggtgcaggcggtgcctttggtgaagctgcaggagacaggggtccctatggcactgacatcggcccaggcggaggctatggggcagcagcagaaggcggcatgtatgctggcaatggcggactattgggagctgactttgctggagatctggattacaatgagctggctgtgagggtgtcagagagcatgcagcgtcagggcctactgcaagggatggcctacactgtccagggcccaccaggccagcctgggccacaggggccacccggcatcagcaaggtcttctctgcctacagcaacgtgactgcggacctcatggacttcttccaaacttatggagccattcaaggaccccctgggcaaaaaggagagatgggcactccaggacccaaaggtgacaggggccctgctgggccaccaggtcatcctgggccacctggccctcgaggacacaagggagaaaaaggagacaaaggtgaccaagtctatgctgggcggagaaggagaagaagtattgctgtcaagccgtgagctagccatggcaggacagctcctggaccaggtctcataatgcatgtggcacttaggtccaaggtctccagagggtgaaagctggagtctgtcaatgtcctactgagacagcacagccaacctagctagcaacatttgttttagtctgaacaatatatacttatagaattcagtcaaagatacacaatctgaaacagcttcatggggtggactctaacagtagttgcaatgttttagaatgagacttacttctctgctatctagatctgaactccttggcttctttacttagttcaagccccagcctaggaaagccagttacataaaagttggctcaggagtcttagagctttacctaaatatgagcccagaaaacggaggatgggggtggggcgccttcctggaggtgacacttgatgggggtgtgttctggttactgttctaaggctgtgccatcagctccttcctcccctgttcattctgcattctctagtcagttggctaagaagtgactcttgcaactaaaaaaattaagaaattcacttcccctctaggaggtgatgatagggtttctaatggttatatgtatatcacattcccatttgcttagaaagtctgattgtagctatgattgtccgtaggcccatactagagttcatggatatgttatactgaaccaggccagagcaaacagaaaaagaaggttgagggcaatggacaaggaaggaataaagggagaagagggaaaacagaaaacctgatgctggggacacagcatcagctcaagacgtcaccctccattctgcactcagaaaatggcacttgggggactgggcgcagttggtctttaaccacttttcaatgtctaaaaacatttgtttgtggtctataagatgaaacatcatttcaatcgtaaaatttcccattaaagaagtttttttttctaa

SEQ ID NO: 3 - DNA, 2350 bp, Homo sapiens
aagcttatattccatgctagggttctggtgttggtgcgtggggttggggtgggactgcagaagtgccttttaagattatgtgattgactgatctgtcattggttccctgccatctttatcttttggattcccctcggaggaggggaggaaggagtttcttttgggttttattgaatcaaatgaaagggaaagtagaggtgttcctatggaggggaggaaggagtttcttttgggttttattgaatcaaatgaaagggaaagtagaggtgttcctatgtcccgggctccggagcttctattcctgggccctgcataagaaggagacatggtggtggtggtggtgggtgggggtggtggggcacagaggaagccgatgctgggctctgcaccccattcccgctcccagatccctctggatatagcaccccctccagtgagcacagcctccccttgccccacagccaacagcaacatgcctcccaacaaagcatctgtccctcagccaaaacccctgttgcctctctctggggaaattgtagggctgggccagggtggggggaccattctctgcagggagattaggagtgtctgtcaggggcgggtggagcggggtggggccctggcttactcacatccttgagagtcctttgctggcagatttggggagcccacagctcagatgtctgtctcagcattgtcttccaagctcctaggccacagtagtggggcgctcccttctctggcttcttctttggtgacagtcaaggtggggttgggggtgacgaagggtcctgcttctcttctaggagcagttgatcccaggaagagcattggagcctccagcaggggctgttggggcctgtctgaggagataggatgcgtcaggcagccccagacacgatcacattcctctcaacatgcctgccggggtctgtggagccgaggggctgatgggagggtggggtgggggccggaagggtttgctttgggaggttgtctgggagattgctgaagttttgatatacacacctccaaagcaggaccaagtggactcctagaaatgtcccctgacccttggggcttcaggagtcagggaccctcgtgtccacctcagccttgcccttgcacagcccagctccactccagcctctactcctccccagaacatctcctgggccagttccacaaggggctcaaacgagggcacctgagctgcccacactagggatgttctgggggtctgagaagatatctggggctggaagaataaaaggcccccctaggcctgttcctggatgcagctccagccactttggggctaagcctgggcaataacaatgccaacgaggcttcttgccatactcggtttacaaaaccctttacatacattgtcgcattggattctcagagctgactgcactaagcagaatagatggtatgactcccactttgcagatgagaacactgaggctcagagaagtgcgaagccctgggtcacagaggcgtaaatgcagagccaggacccacctgaagacccacctgactccaggatgtttcctgcctccatgaggccacctgccctatggtgtggtggatgtgagatcctcaccatagggaggagattagggtctgtgctcagggctggggagaggtgcctggatttctctttgatggggatgttggggtgggaatcacgatacacctgatcagctgggtgtatttcagggatggggcagacttctcagcacagcacggcaggtcaggcctgggagggccccccagacctccttgtctctaatagagggtcatggtgagggaggcctgtctgtgcccaaggtgaccttgccatgccggtgctttccagccgggtatccatcccctgcagcagcaggcttcctctacgtggatgttaaaggcccattcagttcatggagagctagcaggaaactaggtttaaggtgcagaggccctgctctctgtcaccctggctaagcccagtgcgtgggttcctgagggctgggactcccagggtccgatgggaaagtgtagcctgcaggcccacacctccccctgtgaatcacgcctggcgggacaagaaagcccaaaacactccaaacaatgagtttccagtaaaatatgacagacatgatgaggcggatgagaggagggacctgcctgggagttggcgctagcctgtgggtgatgaaagccaaggggaatggaaagtgccagacccgccccctacccatgagtataaagcactcgcatccctttgcaatttacccgagcaccttctcttcactcagccttctgctcgctcgctcacctccctcctctgcaccatgactac

SEQ ID NO: 4 - DNA, 20 bp, mouse collagen 17 forward primer
actcgcctcttcttcaacca

SEQ ID NO: 5 - DNA, 20 bp, mouse collagen 17 reverse primer
gagcaggacgccatgttatt

SEQ ID NO: 6 - DNA, 20 bp, mouse GAPDH forward primer
accacagtccatgccatcac

SEQ ID NO: 7 - DNA, 20 bp, mouse GAPDH reverse primer
tccaccaccctgttgctgta

(19) United States
(12) Patent Application Publication
(10) Pub. No.: US 2009/0215685 A1
(43) Pub. Date: Aug. 27, 2009

(54) METHODS FOR INHIBITING HAIR DEPIGMENTATION AND HAIR LOSS

(75) Inventors: Emi Nishimura, Kanazawa-shi (JP); Hiroshi Shimizu, Sapporo-shi (JP); Shintaro Tanimura, Sapporo-shi (JP); Yuko Tadokoro, Kanazawa-shi (JP); Daisuke Sawamura, Hirosaki-shi (JP); Wataru Nishie, Sapporo-shi (JP)

Correspondence Address: PEARNE & GORDON LLP, 1801 EAST 9TH STREET, SUITE 1200, CLEVELAND, OH, 44114-3108, US

(73) Assignee: National University Corporation Kanazawa University, Kanazawa-shi (JP)

(21) Appl. No.: 12/035,733

(22) Filed: Feb. 22, 2008

(51) Int. Cl. A61K 38/39; A61K 31/711

(52) U.S. Cl. 514/12; 514/44 R; 424/70.13; 424/70.14

(57) ABSTRACT

The present invention aims to provide a method for inhibiting hair depigmentation and a method for inhibiting hair loss, in which hair depigmentation and hair loss are inhibited by collagen XVII.

### BACKGROUND OF THE INVENTION

1. Field of the Invention

The present invention is in the filed of medicinal chemistry and relates to a method for inhibiting hair graying, a method for inhibiting hair depigmentation, a method for inhibiting hair loss, and a method for inhibiting ectopic differentiation of melanocyte stem cell, a method for inhibiting melanocyte stem cell loss, a method for inhibiting hair follicle stem cell abnormal differentiation and a method for inhibiting hair follicle stem cell loss.

2. Related Art

Skin not only protectis an individual from the external environment but plays an important role in the identification of an individual via the pattern and color of the skin and hair. The skin is composed of three layers: the epidermis, the dermis, and subcutaneous adipose tissue; with hair follicles extending continuously from the epidermis towards the subcutaneous adipose tissue. While a constant region of the hair follicle is maintained throughout the hair growth cycle, a transient region of the hair follicle repeats the growth and regression in synchronization with the hair growth cycle. Melanocytes in the skin are responslbe for skin and hair pigmentation. Melanocytes differentiate in the skin to produce melanin, which is supplemented to the hair and skin by being delivered to peripheral keratinocytes, to protect the skin from ultraviolet lights, while simultaneously playing a role in the identification between individuals and protection, etc.

Hair graying is the most obvious sign of ageing that anyone of us experiences. Although the growth of white hair is a widely known phenomenon, in which the number of pigment producing melanocytes of the hair follicles are decreased, very little is known with regard to the mechanism thereof. In 1990, Cotsarelis et al. located hair follicle stem cells in the bulge region of hair follicles. (Cotsarelis G, et al. Label-retaining cells reside in the bulge area of pilosebaceous unit: implications for follicular stem cells, hair cycle, and skin carcinogenesis. Cell 1990; 61(7): 1329-1337). The present inventors are based on previous discovery of melanocyte stem cells (Nishimura E K, et al. Dominant role of the niche in melanoctye stem-cell fate determination. Nature 2002; 416(6883): 854-60.), and proved that the melanocyte stem cell maintenance is essential for melanocyte stem cell maintenance to avoid hair grying (Nishimura E K, et al. Mechanisms of hair graying: incomplete melanocyte stem cell maintenance in the niche. Science 2005; 307(5710): 720-724.)

The number of hair and hair follicles are gradually lost with ageing and male pattern baldness is not the only cause for the ageing-related hair loss. Particularly, male pattern alopecia, which is characterized by frontal and parietal hair follicles regression and resulting hair loss, is known to be under the influence of testosterone (a male hormone), hair tonics targeting the abovementioned male hormone continue to be developed (Japanese Patent Application Laid-Open Publication No. 2004-248632.). In addition to the above-mentioned loss of hair follicles, morphological changes in the connective tissue of skin containing hair follicles also contribute to the hair loss phenomena.

### SUMMARY OF THE INVENTION

Although various methods such as vasodilators and plant extracts, etc., have recently come to be employed in order to inhibit hair depigmentation and hair loss, none of these methods has been effective in solving the abovementioned problems, and therefore, the development of hair loss inhibiting agents and hair depigmentation inhibiting agents is desirable in the pharmaceutically-related and biotechnologically-related product development fields.

Moreover, the mechanism that causes hair loss and hair depigmentation (hair graying) with ageng is unclear. Consequently, scientific break-throughs in the abovementioned mechanisms are desirable in the area of pharmaceutically related and biotechnologically related product development for hair loss treatments or skin or hair regenerative age control, hair-graying treatments, and skin homeostasis.

In view of the abovementioned problems, the present invention aims to provide methods for inhibiting hair depigmentation and hair loss.

Moreover, another aim of the present invention is to elucidate the abovementioned mechanism by providing useful technology for hair loss treatment, hair graying treatment, skin or hair regenerative age control, and skin homeostasis.

In the process of achieving the present invention, the present inventors found that melanocyte stem cells and hair follicle stem cells are prematurely lost with ageing in mice having a collagen XVII genetic mutation (COL17KO mice) prior to premature hair loss and hair graying. Here, it was confirmed that when human collagen XVII was specifically expressed in the region containing the hair follicle stem cells of COL17KO mice, the abnormal differentiation of melanocyte stem cells and hair follicle stem cells was suppressed without any abnormalities in morphology or distribution being observed, such that there was no hair loss or hair graying. Accordingly, the present invention was completed by demonstrating that hair loss and hair graying could be inhibited by collagen XVII.

The present inventors conducted a detailed analysis of collagen XVII expression in the skin to find the region where it is specifically expressed. Moreover, when the observations were focused on the melanocyte stem cells, the ectopic differentiation of melanocyte stem cells into mature melanocytes in the bulge region of COL17KO mice was followed by a subsequent loss of melanocyte stem cells prior to hair graying or hair loss. Furthermore, when the hair follicle stem cells were examined, in addition to the hair follicle stem cells comprising a bulge region of undifferentiated keratinocytes, a loss of hair follicle stem cells via the differentiation and abnormal maturation of undifferentiated keratinocytes comprising the abovementioned bulge region was also observed. Therefore, collagen XVII was essential in the maintenance of melanocyte stem cells and hair follicle stem cells, and an inability to preserve these cells was revealed as the onset of hair graying and hair loss. As a result, the present invention was achieved by demonstrating that collagen XVII mutations caused hair graying resulting in a loss of melanocyte stem cells, that the abnormal differentiation of hair follicle stem cells and melanocyte stem cells was inhibited by collagen XVII, and that hair follicle stem cells and melanocyte stem cells were preserved by the abovementioned loss of melanocyte stem cells.

Specifically, the present invention provides a method for inhibiting hair loss in a mammal in need thereof, comprising administering to the mammal an effective amount of:
(i) collagen XVII containing an amino acid indicated by SEQ ID NO: 1;
(ii) a collagen XVII variant having hair loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair loss inhibiting activity, and encoded by a nucleic acid hybridized under stringent conditions with the nucleotide sequence of SEQ ID NO: 2, or a complimentary strand thereof.

Moreover, the present invention provides a method for inhibiting hair depigmentation in a mammal in need thereof, comprising administering to the mammal an effective amount of:
(i) collagen XVII containing an amino acid indicated by SEQ ID NO: 1;
(ii) a collagen XVII variant having hair depigmentation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair depigmentation inhibiting activity, and encoded by a nucleic acid hybridized under stringent conditions with the nucleotide sequence of SEQ ID NO: 2, or a complimentary strand thereof.

Furthermore, the present invention provides a method for inhibiting hair loss in a mammal in need thereof, comprising administering to the mammal an effective amount of a vector, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair loss inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair loss inhibiting activity, which is hybridized under stringent conditions with the nucleotide sequence of SEQ ID NO: 2 or a complimentary strand thereof, in which the nucleotide sequence is downstream from the promoter sequence. Accordingly, the present invention, as indicated in the below-mentioned examples, inhibits hair loss via collagen XVII.

Moreover, the present invention provides a method for inhibiting hair depigmentation in a mammal in need thereof, comprising administering to the mammal an effective amount of a vector, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair depigmentation inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair depigmentation inhibiting activity, which is hybridized under stringent conditions with the nucleotide sequence of SEQ ID NO: 2 or a complimentary strand thereof, in which the nucleotide sequence is downstream from the promoter sequence. Therefore, the present invention, as indicated in the below-mentioned examples, inhibits hair depigmentation via collagen XVII.

Moreover, the present invention provides a method for inhibiting melanocyte stem cell ectopic differentiation in a mammal in need thereof, comprising administering to the mammal an effective amount of:
(i) collagen XVII containing an amino acid indicated by SEQ ID NO: 1;
(ii) a collagen XVII variant having melanocyte stem cell ectopic differentiation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having melanocyte stem cell ectopic differentiation inhibiting activity, and encoded by a nucleic acid hybridized under stringent conditions with the nucleotide sequence of SEQ ID NO: 2, or a complimentary strand thereof.

Furthermore, the present invention provides a method for inhibiting melanocyte stem cell loss in a mammal in need thereof, comprising administering to the mammal an effective amount of:
(i) collagen XVII containing an amino acid indicated by SEQ ID NO: 1;
(ii) a collagen XVII variant having melanocyte stem cell loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having melanocyte stem cell loss inhibiting activity, and encoded by a nucleic acid hybridized under stringent conditions with the nucleotide sequence of SEQ ID NO: 2, or a complimentary strand thereof. Thus, as indicated in the below-mentioned examples, the present invention inhibits melanocyte stem cell loss via collagen XVII.

Moreover, the present invention provides a method for inhibiting hair follicle stem cell abnormal differentiation in a mammal in need thereof, comprising administering to the mammal an effective amount of:
(i) collagen XVII containing an amino acid indicated by SEQ ID NO: 1;
(ii) a collagen XVII variant having hair follicle stem cell abnormal differentiation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair follicle stem cell abnormal differentiation inhibiting activity, and encoded by a nucleic acid hybridized under stringent conditions with the nucleotide sequence of SEQ ID NO: 2, or a complimentary strand thereof. Thus, as indicated in the below-mentioned examples, the present invention inhibits hair follicle stem cell abnormal differentiation via collagen XVII.

Furthermore, the present invention provides a method for inhibiting hair follicle stem cell loss in a mammal in need thereof, comprising administering to the mammal an effective amount of:
(i) collagen XVII containing an amino acid indicated by SEQ ID NO: 1;
(ii) a collagen XVII variant having hair follicle stem cell loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair follicle stem cell loss inhibiting activity, and encoded by a nucleic acid hybridized under stringent conditions with the nucleotide sequence of SEQ ID NO: 2, or a complimentary strand thereof. Thus, as indicated in the below-mentioned examples, the present invention inhibits hair follicle stem cell loss via collagen XVII.

Moreover, although the abovementioned method for inhibiting hair loss via collagen XVII or a variant thereof, method for inhibiting hair depigmentation via collagen XVII or a variant thereof, method for inhibiting melanocyte stem cell ectopic differentiation via collagen XVII or a variant thereof, method for inhibiting melanocyte stem cell loss via collagen XVII or a variant thereof, method for inhibiting hair follicle stem cell abnormal differentiation via collagen XVII or a variant thereof, and method for inhibiting hair follicle stem cell loss via collagen XVII or a variant thereof provide embodiments of the present invention, the abovementioned constituents may also be optionally combined. Furthermore, the composition for inhibiting hair loss, the composition for inhibiting hair depigmentation, and the medicament for inhibiting hair loss, and the medicament for inhibiting hair depigmentation may have a similar constitution and achieve similar effect.

As indicated in the below-mentioned embodiments, the present invention inhibits hair loss, hair depigmentation, melanocyte stem cell ectopic differentiation, melanocyte stem cell loss, hair follicle stem cell abnormal differentiation, or hair follicle stem cell loss, via human collagen XVII or a variant thereof.

### BRIEF DESCRIPTION OF THE DRAWINGS

FIG. 1 is a serious of photographs/figures showing collagen XVII expression in a control mouse, and hair graying and hair loss as seen in a COL17KO mouse, in which:

FIG. 1a is a series of photographs, showing the progression of hair loss and hair graying in mice at 2 weeks, 6 weeks, 2 months, 3 months, 6 months, and 10 months of age (from top to bottom);

FIG. 1b is photograph of the control mouse at 10 months of age;

FIG. 1c (left-side) is a photograph showing collagen XVII expression in the bulge region and in the basal layer of the epidermis (Sg, sebaceous gland; IFE interfolliculr epidermis; Bg, bulge area);

FIG. 1c (right-side) is a photograph showing a melanocyte stem cell surrounded by a hair follicle stem cell (Sg, sebaceous gland; IFE interfolliculr epidermis; Bg, bulge area);

FIG. 1d is a series of photographs of LacZ staining in the control mice at 6 days, 3 months, and 5 months of age (left to right);

FIG. 1e is a series of photographs of LacZ staining of the knockout mice (KO mouse) at 6 days, 3 months, and 5 months of age (left to right), in which the Lacz-positive melanocyte stem cells gradually decreased, so that the differentiation of melanocyte stem cells became more noticeable by the lightening of the photograph when compared with the control mouse, with an almost complete loss of Lacz-positive cells was seen in mice exceeding 5 months of age;

FIG. 1f shows a RT-PCR band image, in which collagen XVII indicating that there is no collagen XVII expression in GFP-positive melanocytes; and

FIG. 2 is a series of photographs/figures showing a loss of hair follicle stem cells of a mealnocyte stem cell niche, in which:

FIG. 2a shows hyperplasia of the epidermis, and enlarged sebaceous glands in a mouse of approximately 3 months of age (Sg, sebaceous gland; IFE, interfolliculr epidermis);

FIG. 2b shows atrophy of a hair follicle in a mouse of approximately 6 months of age (de, dermis: su, subcutis);

FIG. 2c shows a loss of skin appendages, such as hair follicles in a mouse of approximately 10 months of age;

FIG. 2d shows that CD34, α6 integrin-bipositive hair follicle stem cells were remarkably decreased in the COL17KO mouse;

FIG. 2e shows a stain of the hair follicle stem cell marker from the tissue of the control mouse;

FIG. 2f shows a stain of the hair follicle stem cell marker from the tissue of the COL17KO mouse, in which no keratin 15, CD34, α6 integrin, or S100a6-positive cells were observed; and

FIG. 3 is a series of photographs/figures showing the transgene-mediated correction of COL17 expression in basal keratinocytes rescues MSC loss with normalized c-Myc regulation and skin organization in COL17^{m-/-, h+} ( Col17−/−) mice, K14-hCOL17 transgene was introduced into Col17−/− mice, in which:

FIG. 3a, 3c show the macroscopic phenotype of 6-month-old Col17−/− (a) and Col17−/− mice with K14-hCOL17 transgene.

FIG. 3b, 3d show the expression of human COL17 in skin. Human COL17 is localized in the bulge area (arrows) of rescued mouse hair follicles as well as in the IFE (arrowheads).

FIG. 3e-3h show the distribution and morphology of Dct-lacZ+ melanoblasts in the bulge area (MSCs) (red arrow) are normalized by K14-hCOL17 transgene in Col17−/− mice (Bg; Bulge area).

### DETAILED DESCRIPTION OF THE INVENTION

Here, “hair loss” typically refers to a phenomenon in which there is a gradual decrease in the amount of hair with respect to the originally sufficient amount of hair, and “white/gray hair” refers to a phenomenon in which the amount of white/gray hair gradually increases with respect to hair with original color.

The present inventors first conducted temporal observations of the mice hair, in which detailed observations conducted to determine just when the hair abnormalities began, and whether or not these hair abnormalities occurred after birth, to conclude that hair graying first started as part of the aging process, and progressively continued with hair loss. Furthermore, when a detailed analysis of the expression of collagen XVII in skin was conducted in order to elucidate the mechanism by which hair loss and hair depigmentation accompany aging, the expression of collagen XVII was specifically confirmed in the hair follicle bulge region (the group of cells corresponding to the hair follicle stem cells), as well as the interfollicular epidermis. Moreover, the melanocyte stem cells of COL17KO mice, in which the hair loss and hair graying accompanying aging occurred, were examined. As a result, the ectopic differentiation of melanocyte stem cells into mature melanocytes in the bulge region of COL17KO mice was followed by a subsequent loss of melanocyte stem cells prior to hair graying or hair loss. Furthermore, the abnormalities in the melanocyte stem cells were accompanied by the abnormal differentiation and maturation of undifferentiated keratinocytes constituting the abovementioned bulge region and followint loss of hair follicle stem cells. Moreover, when human collagen XVII employing the promoter (Keratin 14 promoter) was specifically expressed in the hair follicle stem cell containing region of COL17KO mice, since the abnormal differentiation of melanocyte stem cells and hair follicle stem cells was suppressed without any abnormalities in morphology or distribution being observed, it was confirmed that there was no hair loss or hair graying. The abovementioned findings strongly suggest that the development of pharmaceutically-related and biotechnologically-related products targeting skin collagen XVII can be applied in the treatment of white/gray hair or in the treatment of hair loss.

According to the abovementioned results, it is clear that the loss of hair follicle stem cells and melanocyte stem cells occurs from a deficiency in human collagen XVII, which, as a result, leads to hair graying and hair loss. Moreover, it is clear that the loss of hair follicle stem cells and melanocyte stem cells is inhibited by collagen XVII, and that the inhibition of hair graying and hair loss is possible. Furthermore, the present invention was completed under the premise that preserving hair follicle stem cells and melanocyte stem cells by controlling the loss and abnormal differentiation of melanocyte stem cells and hair follicle stem cells via collagen XVII is possible.

Hereinafter, the embodiments of the present invention will be explained in greater detail. The first embodiment of the present invention provides a method for inhibiting hair loss in a mammal in need thereof, comprising administering to the mammal an effective amount of:
(i) collagen XVII containing an amino acid indicated by SEQ ID NO: 1;
(ii) a collagen XVII variant having hair loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair loss inhibiting activity, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant exhibits a hair loss inhibiting function.

The second embodiment of the present invention provides a method for inhibiting hair depigmentation in a mammal in need thereof, comprising administering to the mammal an effective amount of:
(i) collagen XVII containing an amino acid indicated by SEQ ID NO: 1;
(ii) a collagen XVII variant having hair depigmentation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair depigmentation inhibiting activity, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant exhibits a hair depigmentation inhibiting function.

The third embodiment of the present invention provides a method for inhibiting hair loss in a mammal in need thereof, comprising administering to the mammal an effective amount of a vector, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair loss inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair loss inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the collagen XVII or collagen XVII variant to exhibit a hair loss inhibiting function, the vector itself, which expresses collagen XVII or a collagen XVII variant in the region containing the hair follicle stem cells, must also inhibit hair loss.

The fourth embodiment of the present invention provides a method for inhibiting hair depigmentation in a mammal in need thereof, comprising administering to the mammal an effective amount of a vector, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair depigmentation inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair depigmentation inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the collagen XVII or collagen XVII variant to exhibit a hair depigmentation inhibiting function, the vector itself, which expresses collagen XVII or a collagen XVII variant in the region containing the hair follicle stem cells, must also inhibit hair depigmentation.

The fifth embodiment of the present invention provides a method for inhibiting melanocyte stem cell ectopic differentiation in a mammal in need thereof, comprising administering to the mammal an effective amount of:
(i) collagen XVII containing an amino acid indicated by SEQ ID NO: 1;
(ii) a collagen XVII variant having a melanocyte stem cell ectopic differentiation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having a melanocyte stem cell ectopic differentiation inhibiting activity, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant exhibits a melanocyte stem cell ectopic differentiation inhibiting function.

The sixth embodiment of the present invention provides a method for inhibiting differentiation of melanocyte stem cells of the bulge region into the mature melanocytes in a mammal in need thereof, comprising administering to the mammal an effective amount of:
(i) collagen XVII containing an amino acid indicated by SEQ ID NO: 1;
(ii) a collagen XVII variant having an activity, in which the differentiation of melanocyte stem cells of the bulge region into mature melanocytes is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the differentiation of melanocyte stem cells of the bulge region into mature melanocytes is inhibited, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant stimulates melanocyte stem cell maintenance by inhibiting the differentiation of melanocyte stem cells of the bulge region into mature melanocytes or the subsequently occuring loss of melanocyte stem cells.

The seventh embodiment of the present invention provides a method for inhibiting a melanocyte stem cell loss in a mammal in need thereof, comprising administering to the mammal an effective amount of:
(i) collagen XVII containing an amino acid indicated by SEQ ID NO: 1;
(ii) a collagen XVII variant having a melanocyte stem cell loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having a melanocyte stem cell loss inhibiting activity, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant exhibits a melanocyte stem cell loss inhibiting function.

The eighth embodiment of the present invention provides a method for inhibiting a mealnocyte stem cell loss, which occurs subsequent to the differentiation of melanocyte stem cells of the bulge region into mature melanocytes in a mammal in need thereof, comprising administering to the mammal an effective amount of:
(i) collagen XVII containing an amino acid indicated by SEQ ID NO: 1;
(ii) a collagen XVII variant having an activity, in which the melanocyte stem cell loss occurring subsequent to differentiation of the melanocyte stem cells of the bulge region into mature melanocytes is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the melanocyte stem cell loss occurring subsequent to differentiation of the melanocyte stem cells of the bulge region into mature melanocytes is inhibited, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant stimulates melanocyte stem cell maintenance by inhibiting the loss of melanocyte stem cells, which occurs subsequent to the differentiation of melanocyte stem cells of the bulge region into mature melanocytes.

The ninth embodiment of the present invention provides a method for inhibiting hair follicle stem cell abnormal differentiation in a mammal in need thereof, comprising administering to the mammal an effective amount of:
(i) collagen XVII containing an amino acid indicated by SEQ ID NO: 1;
(ii) a collagen XVII variant having a hair follicle stem cell abnormal differentiation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair follicle stem cell abnormal differentiation inhibiting activity, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant stimulates hair follicle stem cell maintenance and/orinhibits of hair follicle stem cell abnormal differentiation.

The tenth embodiment of the present invention provides a method for inhibiting the differentiation of hair follicle stem cells into maturated cells of the epidermis, hair follicles or sebaceous glands in a mammal in need thereof, comprising administering to the mammal an effective amount of:
(i) collagen XVII containing an amino acid indicated by SEQ ID NO: 1;
(ii) a collagen XVII variant having an activity, in which the differentiation of hair follicle stem cells into the maturated cells of the epidermis, hair follicles or sebaceous glands is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the differentiation of hair follicle stem cells into maturated cells of the epidermis, hair follicles or sebaceous glands is inhibited, and encoded by a nucleic acid hybridized under stringent conditions with the nucleotide sequence of SEQ ID NO: 2, or a complimentary strand thereof. As indicated in the below-mentioned examples, hair follicle stem cells consistute the bulge region, and the collagen XVII or collagen XVII variant nhibits the maturation and differentiation of hair follicle stem cells by undifferentiated keratinocytes from an abnormal pathway. As a result, the abnormal differentiation of hair follicle stem cells into maturated cells, and the loss of hair follicle stem cells are inhibited.

The eleventh embodiment of the present invention provides a method for inhibiting hair follicle stem cell loss in a mammal in need thereof, comprising administering to the mammal an effective amount of:
(i) collagen XVII containing an amino acid indicated by SEQ ID NO: 1;
(ii) a collagen XVII variant having a hair follicle stem cell loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair follicle stem cell loss inhibiting activity, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant stimulates hair follicle stem cell maintenance and/or inhibits hair follicle stem cell loss.

The twelfth embodiment of the present invention provides a method for inhibiting the loss of hair follicle stem cells caused by the abnormal maturation and differentiation from the undifferentiated keratinocytes constituting the bulge region of the hair follicle stem cells in a mammal in need thereof, comprising administering to the mammal an effective amount of:
(i) collagen XVII containing an amino acid indicated by SEQ ID NO: 1;
(ii) a collagen XVII variant having an activity, in which the abnormal maturation and differentiation from the undifferentiated keratinocytes constituting the bulge region of the hair follicle stem cells is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the abnormal maturation and differentiation from the undifferentiated keratinocytes constituting the bulge region of the hair follicle stem cells is inhibited, and encoded by a nucleic acid hybridized under stringent conditions with the nucleotide sequence of SEQ ID NO: 2, or a complimentary strand thereof. As indicated in the below-mentioned examples, the collagen XVII or collagen XVII variant inhibits the loss of hair follicle stem cells caused by the maturation and differentiation of undifferentiated keratinocytes constituting the bulge region of the hair follicle stem cells into the cells of the epidermis, sebaceous glands, etc. As a result, the loss of hair follicle stem cells caused by maturation and differention from undifferentiated keratinocytes constituting the bulge region of the hair follicle stem cells is inhibited, and the maintenance of hair follicle stem cells is promoted.

### Collagen XVII

Here, the term “collagen XVII” as employed herein, includes a native sequence collagen XVII. The collagen XVII disclosed herein may also be isolated from various sources, or prepared by a recombinant or synthetic method.

“Native sequence collagen XVII”, is a polypeptide containing an amino acid sequence of a naturally derived collagen XVII. The native sequence collagen XVII may be produced by recombinant or synthetic means, or isolated from various sources.

### Collagen XVII Variant Polypeptide

“Collagen XVII variant” refers to a collagen XVII containing an amino acid sequence that is at least 80% identical to the entire native sequence of collagen XVII disclosed herein. The abovementioned collagen XVII variant includes a collagen XVII in which one amino acid residue or a plurality of amino acid residues (for example, 2, 3 . . . ) of the N- or C-terminus of the entire native amino acid sequence have been added, substituted or deleted. Typically, the collagen XVII variant contains an amino acid sequence that is at least 80% identical with the entire native sequence collagen XVII disclosed herein. However, an amino acid sequence that is at least 90% identical is preferable, and an amino acid sequence that is at least 95% identical is more preferable. Moreover, the amino acid sequence of the entire native sequence collagen XVII is shown by the below-indicated SEQ ID NO: 1.

“Percent (%) amino acid sequence identity” is defined herein as the percentage of amino acid residues in a candidate sequence that are identical with the amino acid residues of the collagen XVII sequence, after aligning the sequences and introducing gaps, if necessary, to achieve the maximum percent sequence identity, without conservative substitutions being considered as part of the sequence identity. The alignment selection for determining the percent of amino acid sequence identity may be a method employed by one who is skilled in the art, such as using conventionally available computer software, for example BLAST, BLAST-2, ALIGN, or Megalign (DNASTAR) software. Moreover, the appropriate parameters for calculating the alignment, including any algorithms necessary for achieving the maximum alignment with respect to entire sequence with which the amino acid is being compared, may be determined by one who is skilled in the art.

### Collagen XVII Variant Polynucleotide

“Collagen XVII variant polynucleotide” is a nucleic acid molecule encoding for a polypeptide with intrinsic polypeptide activity, and having at least 80% sequence identity with respect to the nucleotide sequence encoding for the entire native collagen XVII polypeptide sequence disclosed herein. Typically, the collagen XVII variant polynucleotide has at least 80% nucleic acid sequence identity with respect to the nucleic acid encoding for the entire native collagen XVII polypeptide sequence disclosed herein. However, a nucleic acid sequence identity of at least 90% is preferable, and a nucleic acid sequence identity of at least 95% is more preferable. The variant does not include a native nucleotide sequence. Moreover, the below-indicated SEQ ID NO: 2, does not include the full length sequence encoding for the human collagen XVII.

“Percent (%) nucleic acid sequence identity” for the collagen XVII encoded nucleic acid is defined herein as the percentage of nucleotides in a candidate sequence that are identical with the nucleotides of the collagen XVII polypeptide encoded nucleic acid sequence, after aligning the sequences and introducing gaps, if necessary, to achieve the maximum percent sequence identity. The alignment selection for determining the percent of nucleic acid sequence identity may be a method employed by one who is skilled in the art, such as using conventionally available computer software, for example BLAST, BLAST-2, ALIGN, or Megalign (DNASTAR) software. Moreover, the appropriate parameters for calculating the alignment, including any algorithms necessary for achieving the maximum alignment with respect to entire sequence with which the amino acid is being compared, may be determined by one who is skilled in the art.

In another embodiment of the present invention, the collagen XVII variant polynucleotide is a nucleic acid which encodes the collagen XVII variant polypeptide, and is prefereably a nulceic acid molecule having undergone hybridization with the nucleotide sequence encoding the polypeptide indicated by SEQ ID NO: 1 under purification and stringent hybridization. The collagen XVII variant polypeptide may be encoded by the collagen XVII variant polynucleotide.

### Hair

Here, the term “hair” refers to a skin appendage consisting of a keratin based hair root and hair stem, which includes, for example, head and body hair, etc.

### Hair Loss

Here, the term “hair loss” refers to a portion or all of the hair being loss (falling out), due to aging or disease. In addition to morphological changes in the connective tissue of skin containing hair follicles or an increase in male hormone as some of the various causes of hair loss, the loss of hair follicle stem cells may also play an important role.

### Hair Loss Inhibition

Here, the term “hair loss inhibition” refers to the inhibition of hair loss caused by factors such as morphological changes in the connective tissue of skin, etc., which is different from promoting the extension of short hair, such as hair that has been removed.

### Hair Depigmentation

Here, the term “hair depigmentation” refers to when the color of hair containing the original melanin starts to whiten as a result of a decreased supply of melanin from melanocytes. This includes, for example, graying of body hair or graying of hair on the head.

### Skin Structure

The “skin”, starting from the outermost layer, is divided into three layers: the epidermis, the dermis, and subcutaneous adipose tissue; which covers the entire surface of the body to form a boundary between it and the external envirnoment, and protects internal organs, tissue, etc., from external stimuli or shock. Moreover, the epidermis consists of stratum corneum, stratum granulosum, stratum spinosum, and a stratum basale. The stratum basale is located in the lowermost layer of the epidermis, and forms a wave-like pattern which connects with the dermis. The basal cells of the stratum basale form a monolayer structure consisting of a single row of cuboid or columnar cells, which are interspersed with a plurality of melanocytes. Melanin is produced by melanocytes via stimuli, such as exposure to ultraviolet light, etc. In the stratum basale, the cycle of cell proliferation and new cell formation is repeated continuously via constantly dividing cells that are supplied with nutrients from blood capillaries in hair papilla of the dermis.

### Stem Cells

“Stem cells” are undifferentiated cells that exhibit high self-sustainability (high self-replicability), and are capable of providing differentiated progeny cells. For example, melanocyte stem cells which provide melanocytes as progeny cells, or hair follicle stem cells, which constitute the bulge region as undifferentiated keratinocytes, and differentiate into skin appendages, such the epidermis, sweat glands, and sebaceous glands of the skin, etc., may be exemplified.

### Niche

“Niche” refers to the ecological niche for stem cells. Conventionally, “niche” refers to the location of tissue stem cells. Stem cells are maintained in an undifferentiated state in the niche environment. For example, melanocyte stem cell niche or hair follicle stem cell niche exists in the bulge region. Conventionally, a portion of the progeny cells, which proliferated from the melanocyte stem cells in the bulge region, migrates from the bulge region to the hair matrix, where they maturate and differentiate into melanocytes that supply the hair with melanin.

### Melanocyte Stem Cell Abnormal Differentiation

“Melanocyte stem cell abnormal differentiation” refers to the ectopic (improper) maturation and differentitation of melanocyte stem cells with a dendritic morphology and abundant mature melanosomes, etc., which are usually maintained in an undifferentitated state in the bulge region of a nonaged wild type mouse. The ectopically differentiated cells do not accumulate in hair follicles and are not lost during hair cycle progression. For example, immaturity of the melanocyte stem cells in the bulge region is lost by ageing or COL17 deficency, so that the cells differentiate into mature melanocytes in the bulge region of hair follicles.

### Hair Follicle Stem Cell Abnormal Differentiation

“Hair follicle stem cell abnormal differentiation” refers to the differentiation of hair follicle stem cells along an abnormal differentiation pathway, without sufficient self-maintenance/renewal. For example, the ability to maintain the stem cell properties of the hair follicle stem cells in the bulge region is lost, and abnormal differentiation, such as the differentiation of hair follicle stem cells into hair follicles, the epidermis, or sebaceous glands, etc., is induced.

### Form of Administration

Here, the hair loss inhibiting agent or depigmentation inhibiting agent of the present embodiment may achieve its effects by being administered orally or percutaneously. Moreover, forms of administration may include, for example, a powder, a pill, a tablet, a capsule, a cream, a peroral liquid preparation, or an injection, or an additive added to a hair tonic, a hair lotion, a hair cream, an aerosol, an ointment, a shampoo or rinse, etc.

### Vector

The vector may be any vector that can be conveniently employed in a recombinant DNA procedure. Moreover, the selection of the vector will frequently depend on the compatibility of the vector with the host cell into which the vector is to be introduced. Accordingly, the vector may be an autonomously replicating vector, specifically, a vector existing as extrachromosomal material, the replication of which is independent of chromosomal replication. Examples of the vector may include a plasmid, a cosmid, a phage, a mini-chromosome, or a virus. Alternatively, the vector may be one which, when introduced into a host cell, is integrated into the host cell genome and replicated together with the chromosome(s) into which it has been integrated. Examples of an appropriate vector include bacterial expression vectors and yeast expression vectors. Examples of the abovementioned viral vector may include a vector derived from a virus, such as adenovirus, retrovirus, a papovavirus such as SV40, vaccinia virus, fowl pox virus, or pseudorabies virus, etc. However, the abovementioned viral vector is not particularly limited in any way. Moreover, in the present invention, collagen XVII may be expressed at any arbitrary location depending on the type of expression vector that is constructed.

### Promoter

Here, a promoter is preferably a sequence, which regulates the expression of a polypeptide existing downstream from the promoter sequence itself, and more preferably one that is capable of controlling such an expression in the specific region that contains the hair follicle stem cells. Moreover, although a keratin 5 promoter or a keratin 14 promoter, etc., may be exemplified, in the below-mentioned examples, keratin 14 is employed as the promoter controlling expression in the epidermal basal layer (stratum basale) which contains the hair follicle stem cells, specifically.

### Method of Administration

Here, the method of administration is one by which the abovementioned agent or composition is administered. Thus, the hair loss inhibiting agent or depigmentation inhibiting agent of the present embodiment is thought to achieve its effects by being administered orally or dermally. Moreover, forms of administration may include, for example, a powder, a pill a tablet, a capsule, a cream, a peroral liquid preparation, or an injection, or an additive added to a hair tonic, a hair lotion, a hair cream, an aerosol, an ointment, a shampoo or rinse, etc. Furthermore, the injection may also include a method whereby a subcutaneous injection, etc., is administered by a nurse or doctor.

The administration method, formulation, and dosage of the collagen XVII or the collagen XVII variant in mammals, particularly in human, are described hereafter. The collagen XVII or the collagen XVII variant can be administrated orally, percutaneously or parenterally. Formulations for oral administration include tablets, coated tablets, powder, granules, capsules, microcapsules, and syrups. Formulations for parenteral administration include injectable solutions (including those freeze-dried and dissolved for use), adhesive skin patches, and suppositories. Formulations for percutaneous administration include a powder, a cream, a peroral liquid preparation, orn, or an additive added to a hair tonic, a hair lotion, a hair cream, an aerosol, an ointment, a shampoo or rinse, etc

The collagen XVII or the collagen XVII variant may be administered by any means which acheives inhibiting hair loss or hair depigmentation in a mammal. Preferably, the collagen XVII or the collagen XVII is orally administered. In another embodiment, the collagen XVII or the collagen XVII may be administered as part of an adhesive skin patch. Alternatively, the collagen XVII or the collagen XVII may be formulated into tablets, coated tablets, powder, granules, capsules, microcapsules, and syrups, as the collagen XVII or the collagen XVII in the form of oral formulations is easily administered in mammals, including human beings.

These formulations can be prepared using pharmaceutically acceptable fillers, binders, lubricants, disintegrators, suspending agents, emulsifiers, antiseptic agents, stabilizing agents, and dispersing agents such as lactoses, saccharoses, starches, dextrines, crystalline celluloses, kaolins, calcium carbonate, talc, magnesium stearate, and distilled water or saline. Particular pharmaceutically acceptable components include mannitol, mircocrystalline cellulose, hydroxypropyl cellulose, and magnesium stearate. The dosage varies according to the symptom, age, and body weight of patients.

The collagen XVII or the collagen XVII is preferably administered at an effective oral dosage of 0.0005 mg per kilogram of body weight or higher. In one embodiment, the collagen XVII or the collagen XVII is administered as part of a unitary pharmceutical dosage form containing 5, 10, 15, 20, 25, 30, 35, 40, 45, 50, 55, 60, 65, 70, 75, 80, 85, 90, 95, or 100 mg. When the collagen XVII or the collagen XVII is administered at an effective oral, percutaneous or parenteral dosage of this lower limit or higher, the inhibitory activity in mammals including human beings is improved compared to when lower doses are administered.

### Effects

Hereinafter, the effects of the embodiments will be explained in greater detail.

In the present embodiment, the mechanism of hair loss and hair graying that accompanies aging was elucidated, and experiments were conducted, based on conventionally obtained results, to provide useful technology for hair graying treatment and hair loss treatment. Specifically, as indicated in the examples below, when the melanocytes stem cells and the hair follicle stem cells were examined in detail, it was obvious that there was a loss of melanocyte stem cells and hair follicle stem cells prior to the hair graying and hair loss phenomena which accompanies the aging process. Moreover, it was clear that when a deficiency occurred in human collagen XVII, which is normally expressed in hair follicle stem cells, there was loss of melanocyte stem cells maintained by the hair follicle stem cells, and thus, a subsequent loss of hair follicle stem cells as well. Consequently, it was clear that hair graying and hair loss proceed simultaneously, as a result of stem cells that are no longer capable of supplying differentiated cells.

The method for inhibiting hair loss in a mammal in need thereof of the present embodiment comprises administering to the mammal an effective amount of:
(i) collagen XVII containing an amino acid indicated by SEQ ID NO: 1;
(ii) a collagen XVII variant having hair loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair loss inhibiting activity, and encoded by a nucleic acid hybridized under stringent conditions with the nucleotide sequence of SEQ ID NO: 2, or a complimentary strand thereof. The collagen XVII or collagen XVII variant exhibits the hair loss inhibiting effect indicated in the below-mentioned examples.

Furthermore, the method for inhibiting hair loss of the present embodiment may also be a method for inhibiting hair loss resulting from a loss of hair follicle stem cells in a mammal in need thereof, comprising administering to the mammal an effective amount of:
(i) the collagen XVII containing the amino acid indicated by SEQ ID NO: 1;
(ii) the collagen XVII variant having an activity, in which hair loss resulting from a loss of hair follicle stem cells is inhibited, and containing the amino acid sequence that is at least 80% identical to the amino acid sequence of SEQ ID NO: 1;
(iii) the collagen XVII encoded by the nucleotide sequence indicated by SEQ ID NO:2; or
(iv) the collagen XVII variant having an activity, in which hair loss resulting from a loss of hair follicle stem cells is inhibited, and encoded by the nucleic acid hybridized under stringent conditions with the nucleotide sequence of SEQ ID NO: 2, or the complimentary strand thereof. In such a case, the collagen XVII or collagen XVII variant exhibits the hair follicle stem cell loss inhibiting effect indicated in the below-mentioned examples.

Moreover, the method for inhibiting hair depigmentation in a mammal in need thereof of the present embodiment comprises administering to the mammal an effective amount of:
(i) collagen XVII containing an amino acid indicated by SEQ ID NO: 1;
(ii) a collagen XVII variant having hair depigmentation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair depigmentation inhibiting activity, and encoded by a nucleic acid hybridized under stringent conditions with the nucleotide sequence of SEQ ID NO: 2, or a complimentary strand thereof. In addition, the collagen XVII or collagen XVII variant exhibis the hair depigmentation inhibiting effect indicated in the below-mentioned examples.

Furthermore, the method for inhibiting hair depigmentation of the present embodiment may also be a method for inhibiting hair depigmentation resulting from a loss of melanocyte stem cells in a mammal in need thereof, comprising administering to the mammal an effective amount of:
(i) the collagen XVII containing the amino acid indicated by SEQ ID NO: 1;
(ii) the collagen XVII variant having an activity, in which hair depigmentation resulting from a loss of melanocyte stem cells is inhibited, and containing the amino acid sequence that is at least 80% identical to the amino acid sequence of SEQ ID NO: 1;
(iii) the collagen XVII encoded by the nucleotide sequence indicated by SEQ ID NO:2; or
(iv) the collagen XVII variant having an activity, in which hair depigmentation resulting from a loss of melanocyte stem cells is inhibited, and encoded by the nucleic acid hybridized under stringent conditions with the nucleotide sequence of SEQ ID NO: 2, or the complimentary strand thereof. In such a case, the collagen XVII or collagen XVII variant exhibits the melanocyte stem cell loss inhibiting effect indicated in the below-mentioned examples to thereby result in hair depigmentation inhibiting effect.

Moreover, the method for inhibiting hair loss in a mammal in need thereof of the present embodiment comprises administering to the mammal an effective amount of, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair loss inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair loss inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the the collagen XVII or collagen XVII variant to inhibit hair loss, the vector itself, which expresses the collagen XVII or collagen XVII variant having the abovementioned function in the specific region containing the hair follicle stem cells, must also inhibit hair loss.

Furthermore, the method for inhibiting hair loss in a mammal in need thereof of the present embodiment comprises administering to the mammal an effective amount of a vector, wherein the vector includes a promoter sequence to regulate the expression of collagen XVII or a variant thereof in the stratum basale of the epidermis, and:
(i) a nucleotide sequence encoding for collagen XVII having an amino acid sequence indicated by SEQ ID NO: 1, or a nucleotide sequence encoding for a collagen XVII variant with hair loss inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair loss inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the the collagen XVII or collagen XVII variant to inhibit hair loss, the vector itself, which expresses the collagen XVII or collagen XVII variant having the abovementioned function in the stratum basale of the epidermis, must exhibit a hair loss inhibiting effect.

Moreover, the method for inhibiting hair loss in a mammal in need thereof of the present embodiment may also comprises administering to the mammal an effective amount of a vector, wherein the vector includes a promoter sequence to regulate the expression of collagen XVII or a variant thereof in the cells of stratum basale of the epidermis, and:
(i) a nucleotide sequence encoding for collagen XVII having an amino acid sequence indicated by SEQ ID NO: 1, or a nucleotide sequence encoding for a collagen XVII variant with hair loss inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair loss inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the collagen XVII or collagen XVII variant to inhibit hair loss, the vector itself, which expresses the collagen XVII or collagen XVII variant having the abovementioned function specifically in the cells of the stratum basale of the epidermis, must also exhibit hair loss inhibiting effect.

Furthermore, the method for inhibiting hair depigmentation in a mammal in need thereof of the present embodiment comprises administering to the mammal an effective amount of a vector, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair depigmentation inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair depigmentation inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the collagen XVII or collagen XVII variant to inhibit hair depigmentation, the vector itself, which expresses the collagen XVII or collagen XVII variant having the abovementioned function specifically in the region containing the hair follicle stem cells, must also inhibit hair depigmentation.

Moreover, the method for inhibiting hair depigmentation in a mammal in need thereof of the present embodiment comprising administering to the mammal an effective amount of a vector, wherein the vector includes a promoter sequence to regulate the expression of collagen XVII or a variant thereof in the stratum basale of the epidermis, and:
(i) a nucleotide sequence encoding for collagen XVII having an amino acid sequence indicated by SEQ ID NO: 1, or a nucleotide sequence encoding for a collagen XVII variant with hair depigmentation inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair depigmentation inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the collagen XVII or collagen XVII variant to inhibit hair depigmentation, the vector itself, which expresses the collagen XVII or collagen XVII variant having the abovementioned function in the stratum basale of the epidermis, must exhibit a hair depigmentation inhibiting effect.

Furthermore, the method for inhibiting hair depigmentation in a mammal in need thereof of the present embodiment may also comprise administering to the mammal an effective amount of a vector, wherein the vector includes a promoter sequence to regulate the expression of collagen XVII or a variant thereof in the cells of stratum basale of the epidermis, and:
(i) a nucleotide sequence encoding for collagen XVII having an amino acid sequence indicated by SEQ ID NO: 1, or a nucleotide sequence encoding for a collagen XVII variant with hair depigmentation inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair depigmentation inhibiting activity, which is hybridized under stringent conditions with the nucleotide sequence of SEQ ID NO: 2 or a complimentary strand thereof, in which the nucleotide sequence is downstream from the promoter sequence. Moreover, as indicated in the below-mentioned examples, in order for the composition to inhibit hair depigmentation, the vector itself, which expresses the collagen XVII or collagen XVII variant having the abovementioned function specifically in the cells of the stratum basale of the epidermis, must also exhibit hair depigmentation inhibiting effect.

Furthermore, the method for inhibiting melanocyte stem cell ectopic differentiation in a mammal in need thereof of the present embodiment comprises administering to the mammal an effective amount of:
(i) collagen XVII containing an amino acid indicated by SEQ ID NO: 1;
(ii) a collagen XVII variant having a melanocyte stem cell ectopic differentiation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having a melanocyte stem cell ectopic differentiation inhibiting activity, and encoded by a nucleic acid hybridized under stringent conditions with the nucleotide sequence of SEQ ID NO: 2, or a complimentary strand thereof. Moreover, as indicated in the below-mentioned examples, in order for the the collagen XVII or collagen XVII variant to inhibit melanocyte stem cell ectopic differentiation, the vector itself, which expresses the collagen XVII or collagen XVII variant must also exhibit the melanocyte stem cell ectopic differentiation inhibiting effect, to thereby result in the melanocyte stem cell inhibiting effect.

Moreover, the method for inhibiting melanocyte stem cell ectopic differentiation in a mammal in need thereof of the present embodiment may also be a method for inhibiting the differentiation of melanocyte stem cells of the bulge region into mature melanocytes, comprising administering to the mammal an effective amount of:
(i) collagen XVII containing an amino acid indicated by SEQ ID NO: 1;
(ii) a collagen XVII variant having an activity, in which the differentiation of melanocyte stem cells of the bulge region into mature melanocytes is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the differentiation of melanocyte stem cells of the bulge region into mature melanocytes is inhibited, and encoded by a nucleic acid hybridized under stringent conditions with the nucleotide sequence of SEQ ID NO: 2, or a complimentary strand thereof. In such a case, as indicated in the below-mentioned examples, the collagen XVII or collagen XVII variant inhibits the differentiation of melanocyte stem cells of the bulge region into mature melanocytes, to thereby exhibit a melanocyte stem cell loss inhibiting effect.

Furthermore, the method for inhibiting melanocyte stem cell apoptosis in a mammal in need thereof of the present embodiment comprises administering to the mammal an effective amount of:
(i) collagen XVII containing an amino acid indicated by SEQ ID NO: 1;
(ii) a collagen XVII variant having a melanocyte stem cell loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having a melanocyte stem cell loss inhibiting activity, and encoded by a nucleic acid hybridized under stringent conditions with the nucleotide sequence of SEQ ID NO: 2, or a complimentary strand thereof. Moreover, as indicated in the below-mentioned examples, the loss of melanocyte stem cells is inhibited by the collagen XVII or collagen XVII variant, to thereby result in a melanocyte stem cell maintenance promoting effect.

Moreover, the method for inhibiting melanocyte stem cell apoptosis in a mammal in need thereof of the present embodiment may also be a method for inhibiting melanocyte stem cell loss, in which the inhibition of melanocyte stem cell loss occurs subsequent to the differentiation of melanocyte stem cells of the bulge region into mature melanocytes, comprising administering to the mammal an effective amount of:
(i) collagen XVII containing an amino acid indicated by SEQ ID NO: 1;
(ii) a collagen XVII variant having an activity, in which the inhibition of melanocyte stem cell loss occurs subsequent to the differentiation of melanocyte stem cells of the bulge region into mature melanocytes, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the inhibition of melanocyte stem cell loss occurs subsequent to the differentiation of melanocyte stem cells of the bulge region into mature melanocytes, and encoded by a nucleic acid hybridized under stringent conditions with the nucleotide sequence of SEQ ID NO: 2, or a complimentary strand thereof. In such a case, as indicated in the below-mentioned examples, the collagen XVII or collagen XVII variant inhibits the loss of melanocyte stem cells subsequent to differentiation of the melanocyte stem cells of the bulge region into mature melanocytes, to thereby achieve the melanocyte stem cell loss inhibiting effect.

Furthermore, the method for inhibiting hair follicle stem cell abnormal differentiation in a mammal in need thereof of the present embodiment comprises administering to the mammal an effective amount of:
(i) collagen XVII containing an amino acid indicated by SEQ ID NO: 1;
(ii) a collagen XVII variant having hair follicle stem cell abnormal differentiation inhibiting activity, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair follicle stem cell abnormal differentiation inhibiting activity, and encoded by a nucleic acid hybridized under stringent conditions with the nucleotide sequence of SEQ ID NO: 2, or a complimentary strand thereof. In addition, as indicated in the below-mentioned examples, the collagen XVII or collagen XVII variant promotes hair follicle stem cell maintenance and inhibits hair follicle stem cell abnormal differentiation, to thereby achieve the hair follicle stem cell loss inhibiting effect.

Moreover, the method for inhibiting hair follicle stem cell abnormal differentiation in a mammal in need thereof of the present embodiment may also be a method for inhibiting differentiation of hair follicle stem cells of the bulge region into matured cells, comprising administering to the mammal an effective amount of:
(i) collagen XVII containing an amino acid indicated by SEQ ID NO: 1;
(ii) a collagen XVII variant having an activity, in which the the differentiation of hair follicle stem cells of the bulge region into matured cells is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the the differentiation of hair follicle stem cells of the bulge region into matured cells is inhibited, and encoded by a nucleic acid hybridized under stringent conditions with the nucleotide sequence of SEQ ID NO: 2, or a complimentary strand thereof. Thus, as indicated in the below-mentioned examples, the bulge region consists of hair follicle stem cells, and collagen XVII or collagen XVII variant exhibits a function, in which the abnormal differentiation and maturation of hair follicle stem cells along an abnormal pathway, from undifferentiated keratinocytes, is inhibited, to thereby achieve the hair follicle stem cell loss inhibiting effect.

Furthermore, the method for inhibiting hair follicle stem cell abnormal differentiation in a mammal in need thereof of the present embodiment comprises administering to the mammal an effective amount of:
(i) collagen XVII containing an amino acid indicated by SEQ ID NO: 1;
(ii) a collagen XVII variant having an activity, in which the the differentiation of hair follicle stem cells of the bulge region into matured cells is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which the the differentiation of hair follicle stem cells of the bulge region into matured cells is inhibited, and encoded by a nucleic acid hybridized under stringent conditions with the nucleotide sequence of SEQ ID NO: 2, or a complimentary strand thereof. Moreover, as indicated in the below-mentioned examples, the collagen XVII or collagen XVII variant inhibits the loss of hair follicle stem cells, and promotes the maintenance of hair follicle stem cells.

Moreover, the method for inhibiting hair follicle stem cell loss in a mammal in need thereof of the present embodiment may also be a method for inhibiting hair follicle stem cell loss resulting from the differentiation and maturation of the undifferentiated keratinocytes constituting the hair follicle stem cell bulge region, along an abnormal pathway, into the epidermis, or sebaceous glands, etc., comprising administering to the mammal an effective amount of:
(i) collagen XVII containing an amino acid indicated by SEQ ID NO: 1;
(ii) a collagen XVII variant having an activity, in which hair follicle stem cell loss resulting from the differentiation and maturation of the undifferentiated keratinocytes constituting the hair follicle stem cell bulge region, along an abnormal pathway, into the epidermis, or sebaceous glands, etc., is inhibited, and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having an activity, in which hair follicle stem cell loss resulting from the differentiation and maturation of the undifferentiated keratinocytes constituting the hair follicle stem cell bulge region, along an abnormal pathway, into the epidermis, or sebaceous glands, etc., is inhibited, and encoded by a nucleic acid hybridized under stringent conditions with the nucleotide sequence of SEQ ID NO: 2, or a complimentary strand thereof. Thus, as indicated in the below-mentioned examples, the bulge region consists of hair follicle stem cells, and the collagen XVII or collagen XVII variant exhibits a function, in which the abnormal differentiation and maturation of hair follicle stem cells along an abnormal pathway, from undifferentiated keratinocytes, is inhibited, to thereby achieve the hair follicle stem maintenance promoting effect.

The present invention will be explained with reference to the amino acid sequence indicated by SEQ ID NO: 1, and nucleotide sequence indicated by SEQ ID NO: 2. However, the present invention is not specifically limited to the below-indicated Examples alone, and may be implemented in a variety of forms.

For example, the method for inhibiting hair loss or the method for inhibiting hair depigmentation of the present embodiment, need not use the collagen XVII or a collagen XVII variant alone, and may additionally use any appropriate component that is commonly known to exhibit a hair depigmentation inhibiting effect or hair loss inhibiting effect, or any appropriate component that is commonly known to exhibit an activity that is benefical to skin, especially the scalp, or body hair, especially hair on the head. In such a case, an even more superior hair loss inhibiting effect or depigmentation inhibiting effect is expected, via the additive or synergistic effect between each of the abovementioned components.

For example, a UV blocking agent, such as a sunscreen, etc.; a vitamin (i.e., vitamin A, C or E) or a derivative thereof (i.e., retinyl palmitate, tocopheryl acetate, or tocopheryl palmitate); ceramide; a protein or a protein hydrolysate, peptide, or amino acid (natural); urea or allantoin; a sugar or a sugar derivative, such as a reducing or oxidizing sugar; a plant-derived extract (i.e., and plant extract derived from a member of the family Iridaceae, or a plant extract derived from soybeans), or a microbial-derived extract; a hydroxy acid, especially, hydroxy carboxylic acid or ketocarboxylic acid (i.e., alpha-hydroxy acid, or salicyclic acid), or an ester thereof (i.e., 5-n-octanoyl salicylic acid); diazoxide, spiroxazone, or a phospholipid (i.e., lecithin, especially defatted lecithin); a nicotinic acid derivative (i.e., nicotinic acid ester, especially, tocopheryl nicotinate, benzyl nicotinate, or C1 to C22 alkyl nicotinate, for example, methyl nicotinate or hexyl nicotinate); a pyrimidine derivative (i.e., 2, 4-diamino-6-piperidino pyrimidine-3-oxide, pyrimidine-3-oxide, or 2, 4-diamino pyrimidine-3-N-oxide); an anti-androgen (i.e., a steroidal or non-steroidal 5-α-reductase inhibitor, cyprosterone acetate, azelaic acid and a salt or derivative thereof, flutamide, prostaglandin, for example a salt or ester form PGF-2α or PGE 2, as well as an analog thereof, for example latanoprost; a bactericide, an anti-fungal agent, or an anti-dandruff agent (i.e., a selenium derivative, ketoconazole, octopirox, triclocarban, triclosan, zinc pyrithione, itraconazole, asiatic acid, hinokitiol, mipirocine, tetracycline, especially erythromycin, clinycin hydrochloride, benzoyl peroxide or benzyl peroxide, or minocyclin); calcium channel antagonist, or potassium channel antagonist; a hormone; a steroidal anti-inflammatory agent (i.e., glucocorticoid, corticosteroid), as well as a non-steroidal anti-inflammatory agent (i.e., glycyrrhetinic acid or α-bisabolol, or benzydamine); a retinoid x receptor (RXR) agonist or retinoid antagonist; a free radical scavenger or anti-oxidizing agent (i.e., butylated hydroxyanisole or butylated hydroxytoluene); an anti-seborrheic agent; an anti-parasitic agent; an anti-viral agent; an anti-pruritic agent; a carotenoid (i.e., β-carotene); a lactone or an equivalent salt thereof; an essential fatty acid (i.e., linoleic acid, eicosatetetraenoic acid, linolenic acid, or eicosatrienoic acid), or an ester or amide thereof; an essential oil; phenol or polyphenol, for example a flavonoid; etc.; may be suitably employed along with the collagen XVII or collagen XVII variant.

Moreover, in the method for inhibiting hair depigmentation or hair loss, in which the composition or various agents described above is/are administered, the subject of the administration thereof is not particularly limited in any way, and may be of any race, species, age or sex, so long as the subject is a mammal. However, it is preferable that the composition or various agents described above be administered either therapeutically or prophylactically, particularly, in cases where concern has been raised about the progression of hair depigmentation or hair loss. Moreover, in cases where trends are seen in the progression of hair depigmentation and hair loss in family members, since it is possible that they are either genetically deficient in collagen XVII or a gene variant thereof, or that the gene is not being expressed properly, a specific subject for the administration of the composition or various agents described above is desirable. Furthermore, with regard to the abovementioned subject of administration, according to the results of examinations of the genes that were previously conducted using various conventional methods, a male human was more desirable when confirming whether there was actually a deficiency in collagen XVII or a gene variant thereof, or whether the gene was not being expressed properly.

Furthermore, some preferable ranges of effective dosages are defined in the above embodiments. However, other ranges of effective dosages can be determined for other administration forms. For example, a preferable range of effective dosages for administration by injection can be determined as appropriate. Furthermore, preferable ranges of administration intervals can be determined for particular administration forms in addition to the effective dosages with no more than routine experimentation.

### EXAMPLES

Hereinafter, the present invention will be explained in greater detail with reference to the Example(s). However, the present invention is not specifically limited to the below-mentioned Example(s).

### Example 1

### Analysis of COL17KO Mice

### 1. Experimental Method

### (1) Experimental Animal

The present experiment employed Dct-LacZ transgenic mice, provided by Ian Jackson, Ph.D., MRC Human Genetics Unit, Western General Hospital, Edinburgh, United Kingdom; CAG-CAT-EGFP mice, provided by Jun-ichi Miyazaki, Ph.D., Osaka University, Osaka, Japan; Dct^{tml(Cre)Bee} mice, provided by Friedrich Beermann, Ph.D., Head of the Mutant Mouse Core Facility, ISREC, Swiss Institute for Experimental Cancer Research, Epalinges sur Lausanne, Switzerland; K14-HCOL17 transgenic mice, provided by Kim Yancey, M.D., UT Southwestern Medical School Center, UT Southwestern Medical School, Dallas, Tex., USA; and COL17KO mice, provided by Shimizu Hiroshi, M.D., Ph.D., Hokkaido University, Hokkaido University Graduate School of Medicine, Dermatology Department, Sapporo, Japan.

### (2) Immunohistochemistry

The skin from the dorsal portion of the mouse was fixed in 4% paraformaldehyde, and embedded in OCT compound (Sakura Finetechnical) in a cryomold and frozen or embedded in OCT compound (Sakura Finetechnical) in cryomolds and frozen without being fixed. Next, the skin was cut into 8 μm sections, and blocked with 3% skim milk for 30 minutes at room temperature. Afterwards, the primary antibody was diluted appropriately and reacted overnight at 4° C. Finally, it was purified with PBS, and then, reacted with a secondary antibody.

The primary antibodies employed in the immunostaining were anti-human collagen XVII antibody (N-18, manufactured by Santa Cruz Biotechnology, Inc.); anti-mouse collagen XVII antibody (provided by Toshihiro Tanaka, Ph.D., Department of Dermatology, Shiga University of Medical Science, Ostu, Japan); anti-β-galactosidase antibody (Cappel product, manufactured by MP Biomedicals, Inc.); anti-keratin 15 antibody (manufactured by Covance, Inc.); anti-CD34 antibody (manufactured by eBioscience, Inc.); anti-α6 integrin antibody (manufactured by BD Biosciences Pharmingen); and anti-S100A6 antibody (Lab Vision Corporation). The secondary antibodies employed in the immunostaining were Alexa Flour 488, 568 and 594 antibodies (manufactured by Molecular Probes, Inc.).

### (3) Hematoxylin and Eosin (HE) Staining

The skin from the dorsal portion of the mouse was fixed in Bouin, embedded in paraffin, cut into 4 μm sections, and stained by employing a hematoxylin and eosin (HE) staining method.

### (4) Melanocyte Isolation

Skin was collected from the dorsal portion of a Dct^{tml(Cre)Bee}/CAG-CAT-GFP mouse, in which the melanoctyes containing 6 day old melanocyte stem cells had become GFP-positive, left to react overnight at 4° C. in 1000 U/ml dispase/PBS solution. Afterwards, the dermis was removed under a stereomicriscope. Next, it was supplemented with 0.25% trypsin, left to react for 10 minutes at 37° C., and afterwards, neutralized by fetal bovine calf serum (FCS). Finally, the GFP-positive melanocytes were isolated using a flow cytometry (FACS) method.

### (5) Flow Cytometry (FACS) Analysis

In the isolation of melanocytes the GFP-positive cell fraction was collected by the abovementioned method. In addition, the keratinocytes were reacted in a histological suspension of the abovementioned anti-CD34 antibody and anti-α6 integrin antibody, which was prepared with a similar enzyme treatment. Moreover, a “JSAN”, manufactured by Bay Bioscience; or “FACSCaliber”, manufactured by Becton, Dickinson and Company, was employed in the FACS anaylsis.

(6) Analysis of Mouse Collagen XVII mRNA Expression by RT-PCR

RNA was extracted from the skin of the dorsal region of a mouse and from GFP-positive melanocytes using TRIzol (GIBCO, Invitrogen Corporation). cDna was synthesized by reverse transcription of 3 μg of RNA using a THERMOSCRIPT RT-PCR System (GIBCO, Invitrogen Corporation). The analysis of mouse collagen XVII mRNA expression was conducted using the below-mentioned primer. Moreover, the expression of GAPDH mRNA was measured as an internal standard.

### Mouse Collagen XVII Primer Sequence:

(SEQ ID NO: 4)

| | |
|---|---|
| Forward primer: | 5′-actcgcctcttcttcaacca; |
| and | |

(SEQ ID NO: 5)

| | |
|---|---|
| Reverse primer: | 5′-gagcaggacgccatgttatt. |

### GAPDH:

(SEQ ID NO: 6)

| | |
|---|---|
| Forward primer: | 5′-accacagtccatgccatcac; |
| and | |

(SEQ ID NO: 7)

| | |
|---|---|
| Reverse primer: | 5′-tccaccaccctgttgctgta. |

### Results and Discussion

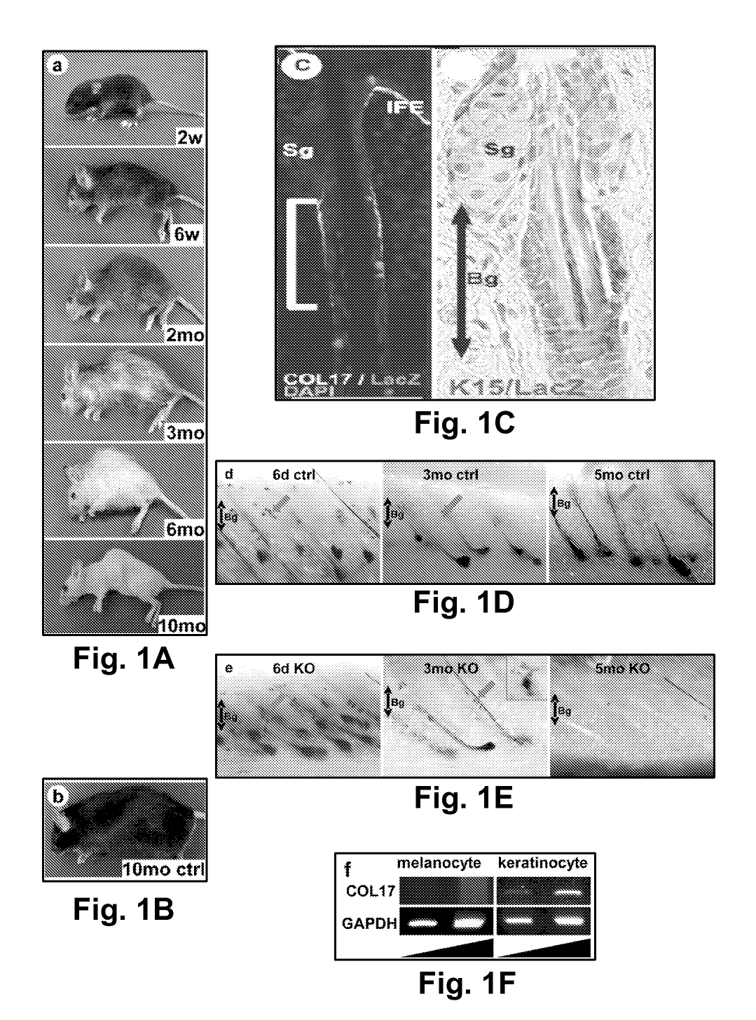

Although, most of COL17KO mice died within two weeks of age, the surviving individuals, which appeared normal until approximately 4 weeks of age, were covered with black body hair (FIG. 1a). However, thereafter, gradually graying was seen. At approximately 3 months of age, graying and hair loss became very noticeable, and body hair was observed as being longer than normal. Moreover, at approximately 6 months of age the entire body of the mice had almost completely whitened. Finally, in mice of approximately 10 months of age, hair loss started occurring throughout the entire body (FIG. 1b).

We confirmed that incomplete maintenance of melanocyte stem cells could be the cause of hair graying. Accordingly, in order to examine whether or not the cause of graying in the COL17KO mice was an inability to maintain melanocyte stem cells, a Dct-LacZ transgene capable of marking melanocyte stem cells was introduced into a COL17KO mouse, and the distribution and morphology of the melanocyte stem cells of the COL17KO mouse was observed. As a result, it was clear that the collagen XVII expression was amplified in the basal cells of the epidermis and the bulge region, and that the melanocyte stem cells were surrounded by hair follicle stem cells (FIG. 1c). Moreover, there were no abnormalities in the melanocyte stem cell distribution and morphology of COL17KO mice immediately after birth. However, in mice of approximately 3 months of age, a gradual decrease in number of melanocytes was observed simultaneously with the presence of maturated melanocytes having a morphology in which differentiated melanocytes containing melanin granules had a plurality of fairly large protrusions (FIGS. 1d and 1e). The melanocyte stem cells were completely loss at approximately 5 months of age when the entire body was white. Consequently, it was concluded that the cause of hair graying in COL17KO mice was an inability to maintain melanocyte stem cells.

Generally, collagen XVII is known to be expressed by epithelial cells forming hemidesmosomes. Therefore, when the RNA of GFP-positive melanocytes of mice expressing GFP in melanocyte specific manner (Dct^{tml(Cre)Bee}/CAG-CAT-GFP mouse) was collected, and the mouse collagen XVII mRNA was analyzed using RT-PCR, in order to examine the presence or absence of collagen XVII expression in melanocytes of the skin containing melanocyte stem cells, it was confirmed that collagen XVII mRNA was not expressed in the melanocytes containing melanocyte stem cells (FIG. 1f). Accordingly, the lack of collagen XVII expression in the abovementioned melanocyte stem cells suggested that the loss of melanocyte stem cells observed in COL17KO mice may be due to an abnormality in the expression of collagen XVII in the cells surrounding the melanocyte stem cells.

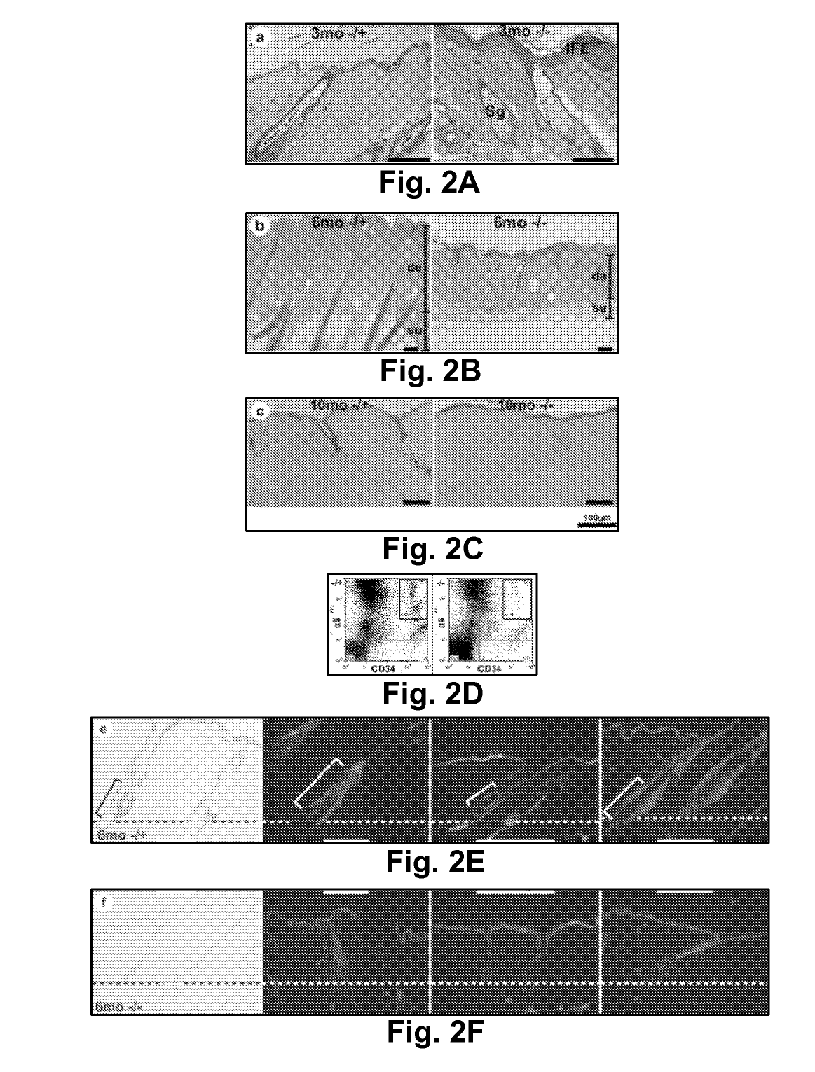

First, the skin of the dorsal region of the COL17KO mice and control mice was checked at weekly age intervals, in order to examine the presence or absence of abnormal hair follicle stem cells. Upon observation of an H.E. stained tissue images, at approximately 3 months of age, enlarged sebaceous glands and hyperplasia of the epidermis were observed (FIG. 2a). Furthermore, at approximately 6 months of age, cystic hair follicles, or atrophied hair follicles were frequently observed (FIG. 2b). Finally, at approximately 10 months of age, appendages, such as hair follicles, were almost completely lost (FIG. 2c). These observations suggest that collagen XVII is necessary in hair follicle maintenance. Moreover, the abovementioned observations of the H.E. stained tissue images were very similar to the H.E. stained tissue images of dermal RacI knockout mice, or basal cell activated c-Myc mice, reported as mice having a depletion of stem cells in the epidermis. Here, since a similar depletion of hair follicle stem cells was also possible in COL17KO mice, staining with an anti-keratin 15 antibody, an anti-CD34 antibody, an anti-α6 integrin antibody, and an anti-S100A6 antibody, which are hair follicle stem cell markers, and FACS analysis were conducted. Whereupon, the depletion of hair follicle stem cells was confirmed (FIG. 2d-2f).

According to the abovementioned results, the Examples clearly show for the first time that collagen XVII deficiency leads to hair graying, and that the deficiency thereof is due to a loss of melanocyte stem cells. Specifically, abovementioned results showed that the collagen VXII of hair follicle stem cells is not only essential in hair follicle stem cell maintenance, but also in melanocyte stem cell maintenance.

In these Examples, although most of COL17KO mice died within the first 2 weeks of age, sufficient follow-up observations revealed that the surviving individuals were covered in black body hair and appeared normal until approximately 4 weeks of age (FIG. 1a). Moreover, subsequent follow-up observations showed that gradual graying was observed, with graying and hair loss becoming very noticeable, and body hair that was longer than normal being seen at approximately 3 months of age. Furthermore, follow-observations made over the course of approximately one year showed that the entire body of the mouse was almost completely whitened by approximately 6 months of age, and that hair loss eventually started to occur throughout the entire body, at approximately 10 months of age. Accordingly, the abovementioned results succeeded in clearly showing for the first time that a collagen XVII deficiency was the cause of mammalian hair loss and hair graying. In previously studies, adequate long-term follow-up observations regarding the distribution and morphology of melanocyte stem cells and the condition of the body hair of COL17KO, K-14 COL17 humanized transgenic mice were not conducted. However, the present Example shows for the first time that hair loss and hair graying can be inhibited by supplying a sufficient amount of collagen XVII in the hair follicle stem cells, in order to prevent a loss of hair follicle stem cells and melanocyte stem cells.

### Example 2

### Analysis of COL17KO, K14-COL17 Humanized Transgenic Mice

### (1) Experimental Animal

COL17KO mice were provided by Shimizu Hiroshi, M.D., Ph.D., Hokkaido University, Hokkaido University Graduate School of Medicine, Dermatology Department, Sapporo, Japan.

### Generation of COL17KO, K14-COL17 Humanized Transgenic Mice

14.7 kb of mouse genomic DNA COL17 fragment was cloned from the mouse 129Sv/Ev genomic library (Stratagene). Next, a 11.5 kb restriction endonuclease NheI to NotI fragment was subcloned. A PGK/NEO cassette was inserted between exon 2 and intron 3 of this fragment to make the targeting vector (Stratagene). The recombinant vector was transfected into 129Sv/Ev embryonic stem cells, then the correctly targeted embryonic stem cell line was micro-injected mouse blastocytes obtained from C57BL/6J mice to generate chimeric mice, which were then mated with C57BL/6J mice. The F1 heterzygotes were crossed with C57BL/6J mice over more than 4 generations, and then intercrossed to generate COL17^{m-/-} mice.

Transgenic mice (C57BL/6J background) expressing the squamous epithelium-specific K14 promoter and human COL17 cDNA (COL17^{m+/+, h+} mice) were crossed with heterozygous COL17^{m-/-} mice, via the incorporation cDNA downstream from the K14 prmoter. Mice that carried both the heterozygous COL17^{m-/-} and the transgene of human COL17 (COL17^{m+/-, h+} mice) were bred to produce COL17KO, K-14 COL17 humanized transgenic mice (COL17^{m-/-, h+} mice).

### (2) Immunohistochemistry

The skin from the dorsal portion of the mouse was immunostained by the same method employed in Example 1. The primary antibodies employed in the immunostaining were anti-mouse collagen XVII antibody (provided by Toshihiro Tanaka, Ph.D., Department of Dermatology, Shiga University of Medical Science, Ostu, Japan), and anti-β-galactosidase antibody (Cappel product, manufactured by MP Biomedicals, Inc.). The Dct stain employed anti-β-galactosidase antibody to stain the skin of a Dct-LacZ transgenic mouse.

### Results and Discussion

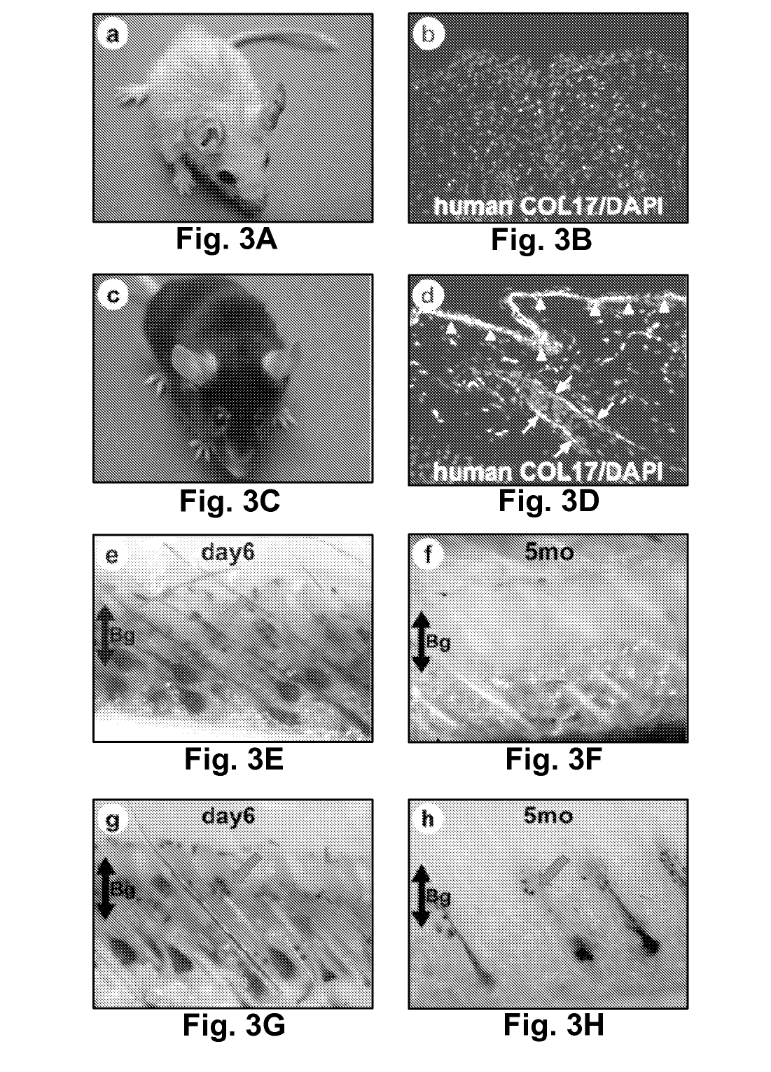

The COL17KO, K-14 COL17 humanized transgenic (COL17^{m-/-, h+}) mice were examined, in order to confirm whether abnormal melanocyte stem cells were caused by abnormal hair follicle stem cells in COL17KO mice. Although human collagen XVII was expressed in the kerantinocytes containing the bulge region in the transgenic mouse, it was confirmed in other cells containing melanocytes that neither the human nor mouse collagen XVII was expressed (FIGS. 3a and 3b (a portion of which is not published)). Not only were hair graying and hair loss not seen in mice exceeding 5 months of age (FIGS. 3a and 3b), but abnormalities in the distribution and morphology of Dct-positive melanocyte stem cells were also not observed (FIGS. 3a and 3d). According to the above mentioned results indicated that collagen XVII deficiency leads to hair graying and hair loss, the hair graying and hair loss therefrom is due to a loss of melanocyte stem cells and/or loss of hair follicle stem cells, and that such a loss of melanocyte stem cells and/or loss of hair follicle stem cells can be prevented by expressing collagen XVII in hair follicle stem cells. Specifically, it was clear that collagen XVII in hair follicle stem cells not only is essential in the maintanence of hair follicle stem cells and melanocyte stem cells, but also is essential in inhibition of hair depigmentation and hair loss.

Specifically, the present Example showed for the first time that when a sufficient amount of collagen was provided in the hair follicle stem cells by expressing collagen XVII in the hair follicle stem cells of mammals that are genetically deficient in collagen XVII, there were no abnormalities in the distribution and morphology of melanocyte stem cells, and no hair graying and hair loss were was observed, even in mice exceeding 5 months of age. In previously studies, adequate long-term follow-up observations regarding the distribution and morphology of melanocyte stem cells and the condition of the body hair of COL17KO, K-14 COL17 humanized transgenic mice were not conducted. However, the present Example shows for the first time that hair loss and hair graying can be inhibited by supplying a sufficient amount of collagen XVII in the hair follicle stem cells, in order to prevent a loss of hair follicle stem cells and melanocyte stem cells.

While preferred embodiments of the present invention have been described and illustrated above, it is to be understood that they are exemplary of the invention and are not to be considered to be limiting. Additions, omissions, substitutions, and other modifications can be made thereto without departing from the spirit or scope of the present invention. Accordingly, the invention is not to be considered to be limited by the foregoing description and is only limited by the scope of the appended claims.

What is claimed is:

1. A method of inhibiting hair loss in a mammal in need thereof, comprising administering to the mammal an effective amount of
(i) collagen XVII containing an amino acid indicated by SEQ ID NO: 1;
(ii) a collagen XVII variant having hair loss inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair loss inhibiting activity, and encoded by a nucleic acid hybridized under stringent conditions with the nucleotide sequence of SEQ ID NO: 2, or a complimentary strand thereof.

2. The method according to claim 1, wherein the hair loss is due to a loss of hair follicle stem cells.

3. A method for inhibiting hair depigmentation in a mammal in need thereof, comprising administering to the mammal an effective amount of
(i) collagen XVII containing an amino acid indicated by SEQ ID NO: 1;
(ii) a collagen XVII variant having hair depigmentation inhibiting activity and containing an amino acid sequence that is at least 80% identical to the amino acid sequence of SEQ ID NO: 1;
(iii) a collagen XVII encoded by a nucleotide sequence indicated by SEQ ID NO:2; or
(iv) a collagen XVII variant having hair depigmentation inhibiting activity, and encoded by a nucleic acid hybridized under stringent conditions with the nucleotide sequence of SEQ ID NO: 2, or a complimentary strand thereof.

4. The method according to claim 3, wherein the hair depigmentation is due to a loss of melanocyte stem cells.

5. A method for inhibiting hair loss in a mammal in need thereof, comprising administering to the mammal an effective amount of a vector, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair loss inhibiting activity, and an amino acid sequence that is at least 80% identical to the amino acid sequence of SEQ ID NO: 1; or
(ii) a nucleotide sequence indicated by SEQ ID NO: 2, or a nucleotide sequence encoding for a collagen XVII variant with hair loss inhibiting activity, which is hybridized under stringent conditions with the nucleotide sequence of SEQ ID NO: 2 or a complimentary strand thereof, wherein
the nucleotide sequence is downstream from the promoter sequence.

6. The method according to claim 5, wherein the promoter regulates expression in the basal layer of the epidermis.

7. The method according to claim 5, wherein the promoter is a keratin 14 promoter with the nucleotide sequence indicated by SEQ ID NO: 3.

8. A method for inhibiting hair depigmentation in a mammal in need thereof, comprising administering to the mammal an effective amount of a vector, wherein the vector includes, wherein the vector includes a promoter sequence to regulate the expression of collagen XVII or a variant thereof in the region containing hair follicle stem cells, and:
(i) a nucleotide sequence encoding for collagen XVII having an amino acid sequence indicated by SEQ ID NO: 1, or a nucleotide sequence encoding for a collagen XVII variant with hair depigmentation inhibiting activity, and an amino acid sequence that is at least 80% identical to the amino acid sequence of SEQ ID NO: 1; or
(ii) a nucleotide sequence indicated by SEQ ID NO:2, or a nucleotide sequence encoding for a collagen XVII variant with hair depigmentation inhibiting activity, which is hybridized under stringent conditions with the nucleotide sequence of SEQ ID NO:2 or a complimentary strand thereof, wherein
the nucleotide sequence is downstream from the promoter sequence.

9. The method according to claim 8, wherein the promoter regulates expression in the basal layer of the epidermis.

10. The method according to claim 8, wherein the promoter is a keratin 14 promoter with the nucleotide sequence indicated by SEQ ID NO: 3.

11. A method for inhibiting melanocyte stem cell ectopic differentiation in a mammal in need thereof, comprising administering to the mammal an effective amount of:
(i) collagen XVII containing an amino acid indicated by SEQ ID NO: 1;
(ii) a collagen XVII variant having melanocyte stem cell ectopic differentiation inhibiting activity and containing an amino acid sequence that is at least 80% identical to the amino acid sequence of SEQ ID NO: 1;
(iii) a collagen XVII encoded by a nucleotide sequence indicated by SEQ ID NO: 2; or
(iv) a collagen XVII variant having melanocyte stem cell ectopic differentiation inhibiting activity, and encoded by a nucleic acid hybridized under stringent conditions with the nucleotide sequence of SEQ ID NO: 2, or a complimentary strand thereof.

12. The method according to claim 11, wherein the melanocyte stem cell ectopic differentiation is differentiation into mature melanocytes in the bulge region.

13. A method for inhibiting melanocyte stem cell loss in a mammal in need thereof, comprising administering to the mammal an effective amount of:
(i) collagen XVII containing an amino acid indicated by SEQ ID NO: 1;
(ii) a collagen XVII variant having melanocyte stem cell loss inhibiting activity and containing an amino acid sequence that is at least 80% identical to the amino acid sequence of SEQ ID NO: 1;
(iii) a collagen XVII encoded by a nucleotide sequence indicated by SEQ ID NO: 2; or
(iv) a collagen XVII variant having melanocyte stem cell loss inhibiting activity, and encoded by a nucleic acid hybridized under stringent conditions with the nucleotide sequence of SEQ ID NO: 2, or a complimentary strand thereof.

14. The method according to claim 13, wherein the melanocyte stem cell loss occurs subsequent to differentiation into mature melanocytes in the bulge region.

15. A method for inhibiting hair follicle stem cell abnormal differentiation in a mammal in need thereof, comprising administering to the mammal an effective amount of:
(i) collagen XVII containing an amino acid indicated by SEQ ID NO: 1;
(ii) a collagen XVII variant having hair follicle stem cell abnormal differentiation inhibiting activity and containing an amino acid sequence that is at least 80% identical to the amino acid sequence of SEQ ID NO: 1;
(iii) a collagen XVII encoded by a nucleotide sequence indicated by SEQ ID NO: 2; or
(iv) a collagen XVII variant having hair follicle stem cell abnormal differentiation inhibiting activity, and encoded by a nucleic acid hybridized under stringent conditions with the nucleotide sequence of SEQ ID NO: 2, or a complimentary strand thereof.

16. The method according to claim 15, wherein the hair follicle stem cells comprise the bulge region as undifferentiated keratinocytes, and the abnormal differentiation of the hair follicle stem cells is abnormal maturation and differentiation of the undifferentiated keratinocytes comprising the bulge region into the epidermis or sebaceous glands, and such.

17. A method for inhibiting hair follicle stem cell loss in a mammal in need thereof, comprising administering to the mammal an effective amount of:
(i) collagen XVII containing an amino acid indicated by SEQ ID NO: 1;
(ii) a collagen XVII variant having hair follicle stem cell cell loss inhibiting activity and containing an amino acid sequence that is at least 80% identical to the amino acid sequence of SEQ ID NO: 1;
(iii) a collagen XVII encoded by a nucleotide sequence indicated by SEQ ID NO: 2; or
(iv) a collagen XVII variant having hair follicle stem cell loss inhibiting activity, and encoded by a nucleic acid hybridized under stringent conditions with the nucleotide sequence of SEQ ID NO: 2, or a complimentary strand thereof.

18. The method according to claim 17, wherein the hair follicle stem cells comprise the bulge region as undifferentiated keratinocytes, and the hair follicle stem cells loss occurs subsequent to the differentiation of hair follicle stem cells of the bulge region into matured cells of the epidermis or sebaceous glands.
